(19) United States
(12) Patent Application Publication
(10) Pub. No.: US 2003/0153006 A1
(43) Pub. Date: Aug. 14, 2003

(54) Novel method for forming polymer pattern

(76) Inventors: Masao Washizu, Kyoto (JP); Osamu Kurosawa, Kyoto (JP)

Correspondence Address: CHRISTIE, PARKER & HALE, LLP, 350 WEST COLORADO BOULEVARD, SUITE 500, PASADENA, CA, 91105, US

(21) Appl. No.: 10/276,906

(22) PCT Filed: May 21, 2001
(86) PCT No.: PCT/JP01/04231

(30) Foreign Application Priority Data
May 22, 2000 (JP) 2000-150065

(51) Int. Cl. C12Q 1/68; G01N 33/53; C07H 21/04; G01N 33/543

(52) U.S. Cl. 435/7.1; 436/518; 530/333; 435/6; 536/25.3

(57) ABSTRACT

A method for forming a combinatorial polymer pattern, which comprises immersing a particulate carrier in monomer addition reactors successively in accordance with a predetermined order using a particle transporting means, to thereby prepare a particle having a polymer having a predetermined sequence bonded thereto, and arranging such particles systematically. The method is practiced by using a plurality of reactors, each of which is provided for a specific monomer, a device for controlling the transfer of particles between the reactors, and a device for arranging particles on a substrate.

### TECHNICAL FIELD

The present invention relates to a novel method for forming a polymer pattern.

### BACKGROUND ART

“DNA chips” employing oligonucleotide patterns on substrates are used as means of detecting specific DNA base sequences. These are based on the following principle. 1) Oligonucleotides (DNA of several to approximately 20 bases) having various sequences are patterned in a two-dimensional array on a substrate, 2) a fluorescent-probe labeled DNA sample is applied onto the chip, and 3) the binding positions are observed under a fluorescent microscope. Since DNA binding occurs in a complementary fashion, binding of the sample DNA at a given position indicates that the sample DNA has a sequence complementary to the sequence immobilized at the bound position. In other words, the sequence of the sample DNA can be determined based on the position of binding on the pattern.

As means of preparing oligonucleotide patterns on substrates there have been developed various methods, including methods of using a metal pin or the like to laboriously coat a glass wafer with each oligonucleotide, ink jet-based methods, methods of accomplishing position-selective chemical synthesis by optical patterning, and the like.

Ordinarily, several hundred or more different oligonucleotides are patterned on a single substrate. In methods based on coating or an ink jet, the actual pattern formation is simple but the number of oligonucleotides to be used must be chemically synthesized in advance, thereby presenting a problem of very high cost. In contrast, methods of direct synthesis on the substrate by optical patterning only require 4 different reagents since they involve polymerization of the 4 different monomers corresponding to the 4 bases A, G, C and T. However, 4 photomasks are necessary for synthesis of each base in the sequence, and therefore synthesis of a 20 mer oligonucleotide, for example, requires the use of 80 photomasks, which not only raises costs but also introduces the complicating technical problem of alignment of the masks. Moreover, the final oligonucleotide purity is as low as about 95% in terms of the yield of synthesis by optical patterning, as compared to a normally high solid-phase synthesis yield of about 99%.

In addition to DNA chips, patterns comprising multiple polypeptides are also prepared on substrates and the binding positions of sample molecules observed, in order to obtain information regarding the affinity between the sample molecules and multiple polypeptides, but the same problems associated with oligonucleotides are also confronted in fabricating the polypeptide patterns that are used for such methods.

### DISCLOSURE OF THE INVENTION

According to the present invention, for fabrication of a pattern of polymers characterized by their monomer sequences, such as oligonucleotides or polypeptides, there are provided solution reactors corresponding to each monomer to be used, and a particle serving as the carrier is successively immersed in each solution reactor in the order of the sequence to be produced, for solid phase synthesis of a polymer on the particle surface. By altering the order of soak it is possible to obtain particles immobilizing polymers of various sequences, which when arranged and immobilized on a wafer permit a polymer pattern to be created on the wafer.

According to a first aspect of the invention there is provided a method for forming a combinatorial polymer pattern whereby different polymers synthesized with a plurality of monomer types in various sequences are immobilized on a wafer in a specific pattern, the method comprising the steps of:

(1) preparing a plurality of reactors each containing a different monomer, a particle transfer control device for controlling transfer of particles between the reactors and a device for arranging the particles on a wafer,

(2) controlling the transport of the particles as carriers by the particle transfer control device for their immersion in the reactors according to a predetermined order, to synthesize polymers having predetermined sequences on the carrier particle surfaces, and

(3) arranging the particles obtained in step (2) on the wafer to form a pattern of polymers having different sequences on the wafer.

According to another aspect of the invention, the polymer is an oligonucleotide.

According to yet another aspect of the invention, the polymer is a peptide.

According to yet another aspect of the invention, the carrier particles are spherical.

According to the first aspect of the invention, the carrier particles are magnetic bodies.

According to a preferred aspect of the invention, the particle transfer is accomplished along a channel or guide groove formed on the substrate.

According to the first aspect of the invention, the particle transfer is accomplished by transporting of the magnetic carrier particles by magnetic force.

According to the first aspect of the invention, there are provided on the substrate branched channels or guide grooves and deflecting devices therein to control movement of the particles, and the order of soak in the reactors is selected by switching the route followed by the particles.

According to the first aspect of the invention, the wafer used has a regular uneven structure for arrangement of the carrier particles on the substrate.

### BRIEF DESCRIPTION OF THE DRAWINGS

FIG. 1 is a schematic illustration showing synthesis of a polymer according to the invention.

FIG. 2 is a schematic illustration showing creation of a polymer pattern according to the invention.

### BEST MODE FOR CARRYING OUT THE INVENTION

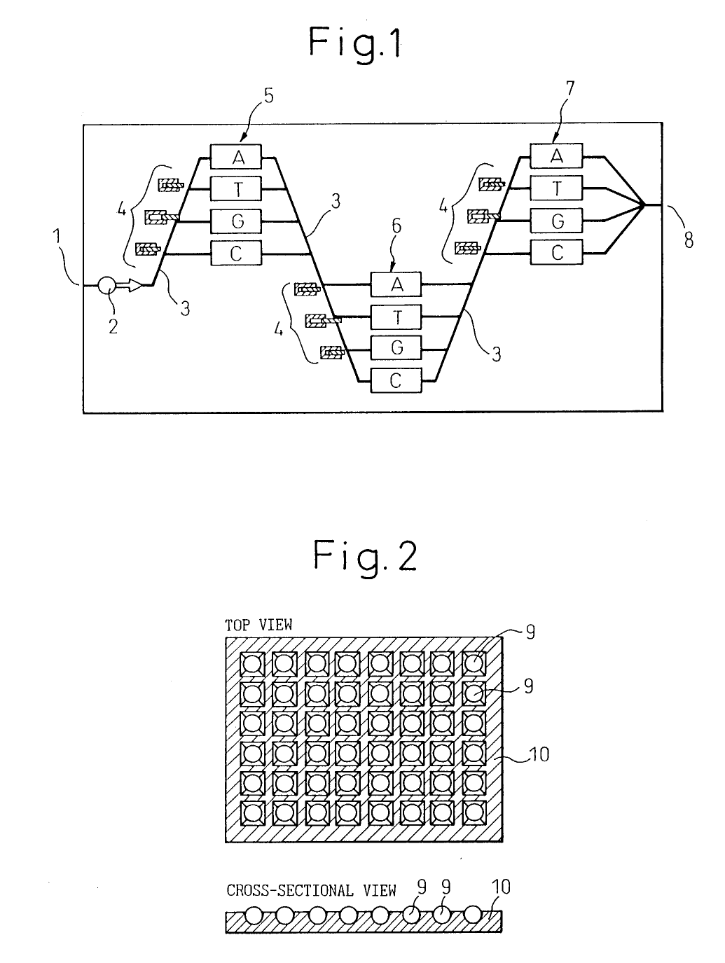

FIG. 1 shows an example of the invention wherein the 4 different monomers A, T, G and C are used for synthesis of a polymer comprising 3 monomers. On a substrate there are provided three sets of reactors each corresponding to the 4 different monomers, and deflecting plate and actuators which control the advancing direction of the carrier particles on a guide groove and groove branches that connect the reactors. Magnetic carrier particles enter through an entry port and roll along the guide groove by the action of a magnet which is provided under the substrate and transported in the right direction of the diagram, causing them to be transported to the right. In the case shown here, the deflecting plate to the left of the G reactor protrudes, causing a particle to be deflected into that groove and enter the reactor, so that G is first added to the particle surface. The exiting particle is then transported again by the magnet to the right along the groove and deflected by a deflecting plate into the T reactor, where T is added. The particle then passes through and out of the G reactor in the same manner, resulting in synthesis of the sequence -G-T-G on the carrier particle surface. By switching deflection by the deflecting plates, highly flexible synthesis can be achieved for a polymer with any desired sequence. Particles immobilizing polymers with various sequences obtained in this manner may be anchored in an arranged manner on a wafer to obtain a polymer pattern on the wafer.

The carrier particles used here are, for example, magnetic stainless steel spherical particles with a diameter of 0.5-1 mm which roll easily and are readily driven by a magnetic field, but there is no restriction to such particles.

In the example shown in FIG. 1, a polymer of any desired length may be synthesized by aligning more than 3 polymer reactors. Also, while the number of reactors provided corresponds to the polymer length in this example in order to explain the movement of the particles in brief, exactly the same effect can be achieved with a construction wherein only one set of reactors is provided and the particles are transported between them in the prescribed order.

FIG. 2 is an example of a polymer pattern arrangement according to the invention, obtained by bonding particles with various monomer sequences created in the manner shown in FIG. 1 on a wafer with a regular uneven structure.

According to the polymer synthesis and polymer pattern formation method of the invention, it is possible to synthesize polymers in a highly flexible manner by combinations of reagents in the number of the types of monomers used, and obtain therefrom polymer patterns arranged at specific positions on a wafer.

List of Reference Numerals

1 Carrier particle entry port

2 Carrier particle

3 Guide grooves

4 Deflecting plates and actuators

5 Reactor set (Set 1)

6 Reactor set (Set 2)

7 Reactor set (Set 3)

8 Carrier particle exit port

9 Carrier particle with specific immobilized sequence

10 Wafer with uneven structure

1. A method for forming a combinatorial polymer pattern whereby different polymers synthesized with a plurality of monomer types in various sequences are immobilized on a wafer in a specific pattern, the method comprising the steps of:
(1) preparing a plurality of reactors each containing a different monomer, a particle transfer control device for controlling transfer of particles between the reactors and a device for arranging the particles on a wafer,
(2) controlling the transport of the particles as carriers by said particle transfer control device for their immersion in the reactors according to a predetermined order, to synthesize polymers having predetermined sequences on the carrier particle surfaces, and
(3) arranging the particles obtained in step (2) on a wafer to form a pattern of polymers having various sequences on said wafer.

2. A method for forming a polymer pattern according to claim 1, wherein said polymer is an oligonucleotide.

3. A method for forming a polymer pattern according to claim 1, wherein said polymer is a peptide.

4. A method for forming a polymer pattern according to claim 1, wherein said carrier particles are spherical.

5. A method for forming a polymer pattern according to claim 1, wherein said carrier particles are magnetic bodies.

6. A method for forming a polymer pattern according to claim 1, wherein said particle transfer is accomplished along a channel or guide groove formed on the substrate.

7. A method for forming a polymer pattern according to claim 1, wherein said particle transfer is accomplished by transporting of the magnetic carrier particles by magnetic force.

8. A method for forming a polymer pattern according to claim 1, wherein there are provided on said substrate branched channels or guide grooves and deflecting devices therein to control movement of the particles, and the order of soak in the reactors is selected by switching the route followed by the particles.

9. A method for forming a polymer pattern according to claim 1, wherein the wafer used has a regular uneven structure for arrangement of said carrier particles on the wafer.
